(19) United States
(12) Patent Application Publication
(10) Pub. No.: US 2022/0127298 A1
(43) Pub. Date: Apr. 28, 2022

(54) THERMOSPOROTHRIX HAZAKENSIS-DERIVED ANTIBACTERIALLY ACTIVE SUBSTANCE

(71) Applicant: KENNAN EISEI KOGYO CO., LTD., Shibata-gun, Miyagi (JP)

(72) Inventors: Hideaki TAKANO, Fujisawa-shi, Kanagawa (JP); Minato SATO, Fujisawa-shi, Kanagawa (JP); Shoichi AMANO, Fujisawa-shi, Kanagawa (JP); Hatsumi TAKANO, Fujisawa-shi, Kanagawa (JP); Kenji UEDA, Fujisawa-shi, Kanagawa (JP); Kazuo FURIHATA, Tokyo (JP); Shohei SAKUDA, Utsunomiya-shi, Tochigi (JP); Akira YOROTA, Osaka (JP); Shuhei YABE, Shibata-gun, Miyagi (JP)

(73) Assignee: KENNAN EISEI KOGYO CO., LTD., Shibata-gun, Miyagi (JP)

(21) Appl. No.: 17/428,109

(22) PCT Filed: Feb. 4, 2020
(86) PCT No.: PCT/JP2020/004076

§ 371 Date: Aug. 3, 2021

(30) Foreign Application Priority Data
Feb. 4, 2019 (JP) PCT/JP2019/003813
Jul. 26, 2019 (JP) 2019-138058

(51) Int. Cl. C07H 15/244; A61K 35/74; A61K 31/704; A01N 43/08; A01N 63/20

(52) U.S. Cl. CPC C07H 15/244; A61K 35/74; A01N 63/20; A01N 43/08; A61K 31/704

(57) ABSTRACT

This invention provides a novel compound having an antibacterial activity, and a method for producing the same. The compound is represented by Formula (I):

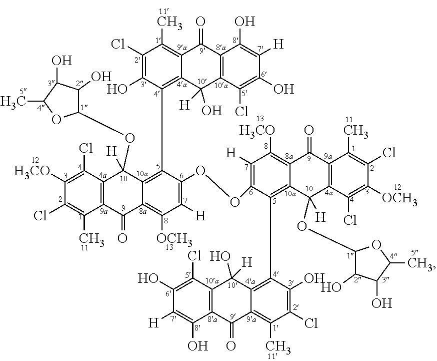 [SMILES: COc1cc(OOc2cc(OC)c3c(c2-c2c(O)c(Cl)c(C)c4c2C(O)c2c(Cl)c(O)cc(O)c2C4=O)C(OC2OC(C)C(O)C2O)c2c(Cl)c(OC)c(Cl)c(C)c2C3=O)c(-c2c(O)c(Cl)c(C)c3c2C(O)c2c(Cl)c(O)cc(O)c2C3=O)c2c1C(=O)c1c(C)c(Cl)c(OC)c(Cl)c1C2OC1OC(C)C(O)C1O]

or a salt or ester thereof.

### TECHNICAL FIELD

The present invention relates to a novel compound having an antibacterial activity, and a method for producing the same.

### BACKGROUND ART

Thermosporothrix hazakensis (herein also referred to as “T. hazakensis”) is a bacterium belonging to the class Ktedonobacteria within the order Ktedonobacterales in the phylum Chloroflexi, and an aerobic Gram-positive bacterium. The present inventors have isolated a T. hazakensis SK20-1^{T} strain (=NBRC 105916T=JCM 16142T=ATCC BAA-1881T) and demonstrated that the same has the ability to decompose cellulose, xylan, and chitin (Non Patent Literature 1). So far, some antibacterial active substances originated from T. hazakensis have been reported (Non-Patent Literature 2 and 3), but additional antibacterial active substances have been desired.

### CITATION LIST

### Non-Patent Literature

Non-Patent Literature 1: S. Yabe, et al., International Journal of Systematic and Evolutionary Microbiology (2010), 60, 1794-1801
Non-Patent Literature 2: J. S. Park, et al., Chem Bio Chem (2014), 15, 527-532
Non-Patent Literature 3: J. S. Park, et al., The Journal of Antibiotics (2015), 68, 60-62

### SUMMARY OF INVENTION

### Technical Problem

An object of the present invention is to provide a novel compound having an antibacterial activity, and a method for producing the same.

### Solution to Problem

As a result of diligent studies to achieve the above object, the present inventors have found a novel antibacterial active substance from the T. hazakensis SK20-1^{T} strain (=NBRC 105916T=JCM 16142T=ATCC BAA-1881T), thereby completing the present invention.

The present invention includes the following inventions.

(1) A compound represented by Formula (I):

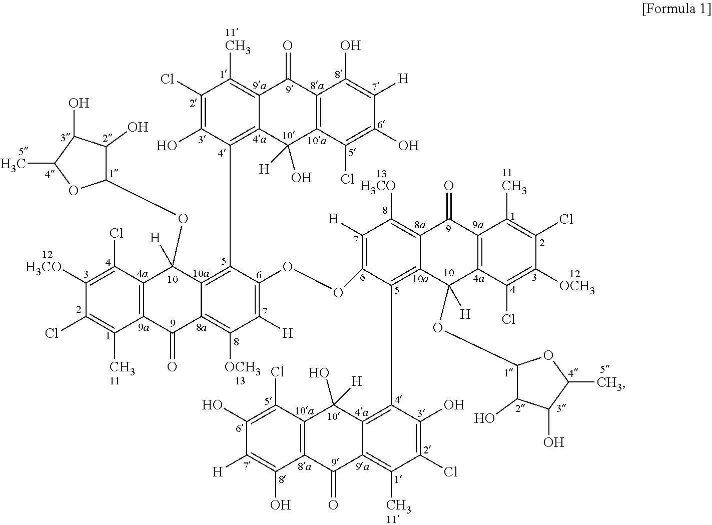 [SMILES: COc1cc(OOc2cc(OC)c3c(c2-c2c(O)c(Cl)c(C)c4c2C(O)c2c(Cl)c(O)cc(O)c2C4=O)C(OC2OC(C)C(O)C2O)c2c(Cl)c(OC)c(Cl)c(C)c2C3=O)c(-c2c(O)c(Cl)c(C)c3c2C(O)c2c(Cl)c(O)cc(O)c2C3=O)c2c1C(=O)c1c(C)c(Cl)c(OC)c(Cl)c1C2OC1OC(C)C(O)C1O]

or a salt or ester thereof.

(2) A compound having an absorption maximum at 340 nm in its UV spectrum and a molar absorption coefficient of 30630;

having a molecular formula of C₇₄H₅₈Cl₈O₂₆ as determined by analyses of an ESI/TOF/MS spectrum and a high-resolution mass spectrum;

showing signals at δ11.49, 10.92, 6.67, 6.41, 6.21, 6.00, 5.91, 4.95, 4.26, 3.96, 3.95, 3.79, 3.71, 3.70, 2.69, 2.19, and 0.90 in a ¹H NMR spectrum (DMSO-d₆); and

showing signals at δ188.61, 184.49, 162.20, 159.96, 158.11, 155.76, 155.25, 150.91, 140.73, 140.25, 140.25, 138.20, 137.79, 137.26, 134.60, 133.53, 132.77, 125.42, 121.51, 120.24, 119.19, 114.71, 111.89, 109.54, 102.99, 99.78, 98.55, 77.15, 72.75, 70.21, 67.00, 61.20, 60.68, 56.94, 17.83, 15.90, and 14.96 in a ¹³C NMR spectrum (DMSO-d₆);

or a salt or ester thereof.

(3) The compound, or a salt or ester thereof according to (2) above, obtained by culturing Thermosporothrix hazakensis in a culture medium, and recovering the compound, or a salt or ester thereof from the culture product.

(4) The compound, or a salt or ester thereof according to (3) above, wherein the compound, or a salt or ester thereof is recovered by treating an ethyl acetate extract with a silica gel column, the ethyl acetate extract being obtained by partitioning an aqueous acetone solution extract of the culture product between ethyl acetate and water.

(5) The compound, or a salt or ester thereof according to (3) or (4) above, wherein Thermosporothrix hazakensis is a Thermosporothrix hazakensis SK20-1^{T} strain.

(6) A method of producing the compound, or a salt or ester thereof according to any one of (1) to (5) above, comprising a step of culturing Thermosporothrix hazakensis in a culture medium, and recovering the compound, or a salt or ester thereof according to any one of (1) to (5) above from the culture product.

(7) The method according to (6) above, wherein the compound, or a salt or ester thereof according to any one of (1) to (5) above is recovered by treating an ethyl acetate extract with a silica gel column, the ethyl acetate extract being obtained by partitioning an aqueous acetone solution extract of the culture product between ethyl acetate and water.

(8) The method according to (6) or (7) above, wherein Thermosporothrix hazakensis is a Thermosporothrix hazakensis SK20-1^{T} strain.

(9) An antibacterial agent comprising the compound, or a salt or ester thereof according to any one of (1) to (5) above, as an active ingredient.

This description includes the disclosure contents of International Application PCT/JP2019/003813 and Japanese Patent Application No. 2019-138058, based on which the present application claims for priority.

### Advantageous Effects of Invention

The present invention provides a novel antibacterial active substance originated from a T. hazakensis SK20-1^{T} strain (=NBRC 105916T=JCM 16142T=ATCC BAA-1881T), and a method for producing the same.

### BRIEF DESCRIPTION OF DRAWINGS

FIG. 1-1 shows the ¹H-NMR spectrum of HK-1.

FIG. 1-2 shows the ¹³C-NMR spectrum of HK-1.

FIG. 1-3 shows the COSY spectrum of HK-1.

FIG. 1-4 shows the HSQC spectrum of HK-1.

FIG. 1-5 shows the HMBC spectrum of HK-1.

FIG. 1-6 (a) shows the 1D-ROESY spectrum of HK-1 radiated at 2.19 ppm, and FIG. 1-6 (b) is an enlarged view thereof.

FIG. 1-7 (a) shows the 1D-ROESY spectrum of HK-1 irradiated at 6.21 ppm, and FIG. 1-7 (b) is an enlarged view thereof.

FIG. 1-8 shows the results of the NMR spectrum analysis of HK-1.

FIG. 1-9 shows a ¹³C-NMR spectrum for observing the isomer shift caused by deuterium substitution of HK-1.

FIG. 2 shows the monomer structure of HK-1.

FIG. 3 shows the planar structure of HK-1.

FIG. 4-1 shows the results of a measurement of the antibacterial activity of HK-1 heat-treated at a high temperature after a pH change.

FIG. 4-2 shows the results of a measurement of the antibacterial activity against B. subtilis overtime due to change in pH and storage temperature.

FIG. 4-3 shows the results of measurement of the antibacterial activity against G. stearothermophilus overtime due to change in pH and storage temperature.

### DESCRIPTION OF EMBODIMENTS

The present invention relates to a compound represented by Formula (I), or a salt or ester thereof (hereinafter, also referred to as the compound of the present invention).

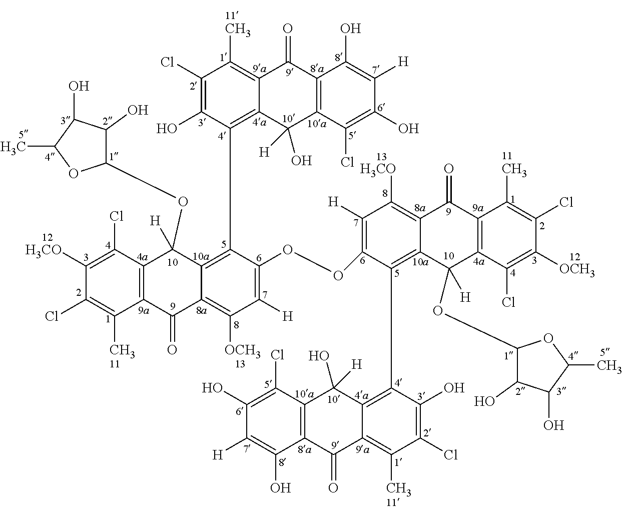

The compound of the present invention has a high antibacterial activity. The compound of the present invention has a high antibacterial activity, especially with respect to Gram-positive bacteria.

The compound of Formula (I) has a structure in which the following structure are dimerized.

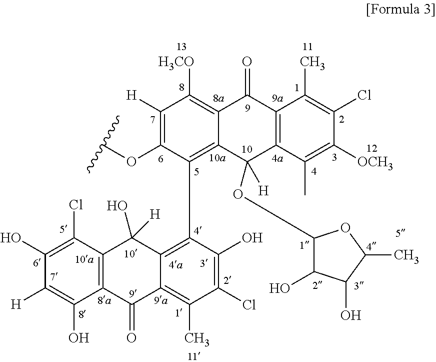 [SMILES: *Oc1cc(OC)c2c(c1-c1c(O)c(Cl)c(C)c3c1C(O)c1c(Cl)c(O)cc(O)c1C3=O)C(OC1OC(C)C(O)C1O)c1c(C)c(OC)c(Cl)c(C)c1C2=O]

The dimer is constructed by forming a peroxide via the oxygen at C-6 position. When an asymmetric carbon is present in the compound of Formula (I), all the enantiomers and mixtures thereof are included, and when a stereoisomer is present, all the stereoisomers and mixtures thereof are included. The compound of Formula (I) may form a hydrate, a solvate, or a crystal polymorph. The glycosidic linkage at C-10 position may be either α-linkage or β-linkage.

In addition, the present invention also relates to a compound having physico-chemical properties, that is:

the compound has an absorption maximum at 340 nm in its UV spectrum, and a molar absorption coefficient of 30630;

it has a molecular formula of C₇₄H₅₈Cl₈O₂₆ as determined by analyses of an ESI/TOF/MS spectrum and a high-resolution mass spectrum;

it shows signals at δ11.49, 10.92, 6.67, 6.41, 6.21, 6.00, 5.91, 4.95 (d, 4.7), 4.26, 3.96, 3.95, 3.79, 3.71, 3.70, 2.69, 2.19, and 0.90 (d, 5.7) in a ¹H NMR spectrum (DMSO-d₆, J=Hz); and

it shows signals at δ188.61, 184.49, 162.20, 159.96, 158.11, 155.76, 155.25, 150.91, 140.73, 140.25, 140.25, 138.20, 137.79, 137.26, 134.60, 133.53, 132.77, 125.42, 121.51, 120.24, 119.19, 114.71, 111.89, 109.54, 102.99, 99.78, 98.55, 77.15, 72.75, 70.21, 67.00, 61.20, 60.68, 56.94, 17.83, 15.90, and 14.96 in a ¹³C NMR spectrum (DMSO-d₆); or a salt, or an ester thereof. Possession of such physico-chemical properties has been demonstrated in the following Examples. The compound having such physico-chemical properties, or a salt or ester thereof, has a high antibacterial activity. The compound having such physico-chemical properties, or a salt or ester thereof, has a high antibacterial activity particularly with respect to Gram-positive bacteria. When an asymmetric carbon is present in the compound having such physico-chemical properties, or a salt or ester thereof, all the enantiomers and mixtures thereof are included, and when a stereoisomer is present, all the stereoisomers and mixtures thereof are included. The compound having such physico-chemical properties, or a salt or ester thereof, may form a hydrate, a solvate, or a crystal polymorph. The compound of Formula (I) and the compound having such physico-chemical properties, and a salt or ester thereof, are also collectively referred to as the compound of the present invention.

There is no particular restriction on the salt of the compound of Formula (I) or the compound having the above physico-chemical properties, and various salts can be used. Examples thereof include an alkali metal salt, such as a sodium salt, and a potassium salt, a salt of an alkaline earth metal, such as calcium and magnesium, and an organic amine salt, such as an ammonium salt, a triethanolamine salt, and a triethylamine salt. In addition, these salts may form a hydrate, a solvate, or a crystal polymorph.

There is no particular restriction on the ester of the compound of Formula (I) or the compound having the above physico-chemical properties, and examples thereof include an ester with a fatty acid such as acetic acid, and an aromatic carboxylic acid such as benzoic acid. The fatty acid includes a saturated fatty acid and an unsaturated fatty acid, and the chain length thereof may be short chain, medium chain, and long chain, and specific examples thereof include, but not limited to, acetic acid, propionic acid, butyric acid, caproic acid, capric acid, lauric acid, myristic acid, palmitic acid, stearic acid, arachidic acid, behenic acid, lignoceric acid, decenoic acid, oleic acid, erucic acid, linoleic acid, linolenic acid, arachidonic acid, and eicosapentaenoic acid. The aromatic fatty acid includes benzoic acid, a benzoic acid having a substituent, such as an amino group, and a hydroxy group at ortho-, meta-, and/or para-position, and also a polycyclic aromatic carboxylic acid such as naphthoic acid. The compound of Formula (I) can form ester(s) with 1 to 12 hydroxy groups in Formula (I), preferably with hydroxy group(s) bonded to at least one carbon at a position selected from C-3′, C-6′, C-8′, C-10′, C-2″, and C-3″. These esters may be prepared by reacting the compound of Formula (I) or the compound having the above physico-chemical properties with a corresponding carboxylic acid in the usual manner, for example, using an acidic catalyst.

The present invention also relates to a method for producing the compound of Formula (I) or the compound having the above physico-chemical properties, or a salt or ester thereof (hereinafter also referred to as the production method of the present invention), comprising a step of culturing Thermosporothrix hazakensis in a culture medium, and recovering the compound of Formula (I) or the compound having the above physico-chemical properties, or a salt or ester thereof, from the culture product. According to the production method of the present invention, the compound of the present invention can be obtained at a high yield.

For the culture of T. hazakensis in the production method of the present invention, an ordinary method for culturing microorganisms is used. As the culture medium either of a synthetic culture medium or a natural culture medium may be used insofar as it contains appropriately a utilizable carbon source, nitrogen source, inorganic substance, and necessary growth and production promoting substance. As a carbon source, glucose, starch, dextrin, mannose, fructose, maltose, lactose, molasses, etc. are used singly or in combination. Further, if necessary, a hydrocarbon, an alcohol, an organic acid, an amino acid (e.g. tryptophan) and the like may also be used. As a nitrogen source, ammonium chloride, ammonium sulfate, ammonium nitrate, sodium nitrate, urea, peptone, a meat extract, a yeast extract, dried yeast, a corn steep liquor, soybean flour, cottonseed cake, casamino acid, etc. are used singly or in combination. Further, an inorganic salt, such as common salt, sodium chloride, potassium chloride, magnesium sulfate, calcium carbonate, potassium dihydrogen phosphate, dipotassium hydrogen phosphate, ferrous sulfate, calcium chloride, manganese sulfate, and zinc sulfate may be added as necessary. Further, trace components that promote growth of a microorganism to be used and the production of the compound of the present invention may be appropriately added, and those skilled in the art can select appropriate components.

Culturing of T. hazakensis in such a nutrient culture medium can be carried out in the same way as generally used in the production of antibiotics by microorganisms. Usually, culture under aerobic conditions is preferable, and usually it can be carried out with stirring and/or aeration. As a culture method, any of static culture, shaking culture, and liquid culture with aeration stirring can be used, and shaking culture is suitable.

There is no particular restriction on the applicable culture temperature, insofar as growth of T. hazakensis is not substantially inhibited and it may be appropriately selected within a range suitable for producing the antibiotic substance. For example, the culture temperature in a range of 35 to 50° C. is particularly preferred. Culture in a culture medium with a pH of 3 to 11 is possible, and a pH of 6 to 10 is preferable. The culture can be usually be continued until a sufficient amount of an antibiotic substance has accumulated. Although such a culture time varies depending on the composition of the culture medium, culture temperature, operating temperature, production strain, etc., usually a target antibiotic substance can be produced and accumulated in the culture solution and bacterial body by culturing of 2 to 8 days

The accumulated amount of a novel antibiotic in a culture product may be quantitatively determined by a paper disk method used commonly for an activity test of an antibiotic using a Geobacillus stearothermophilus ATCC 7953 strain and/or a Bacillus subtilis NBRC 3134^{T} strain as an assay strain.

A novel antibiotic accumulated in the culture product and bacterial body is recovered from the culture product. After culturing, if necessary, the bacterial body and the supernatant are separated by a separation method per se publicly known such as filtration and centrifugation, and then the novel antibiotic can be recovered by isolating and purifying it from the culture supernatant using solvent extraction using an organic solvent, especially acetone or the like, chromatography, utilizing adsorption or ion exchange capacity, gel filtration, and chromatography utilizing liquid-liquid partition singly or in combination. As the carrier for chromatography having adsorption or ion exchange capacity, activated carbon, silica gel, a porous styrene/divinylbenzene copolymer resin, or various ion exchange resins can be used. The bacterial body is extracted by 50% acetone and then isolated and purified similarly to the supernatant using various chromatographies singly or in combination. A novel antibiotic is preferably recovered by treating with a silica gel column an ethyl acetate extract obtained by partitioning an aqueous acetone solution extract of the culture product between ethyl acetate and water. It is preferable to use ethyl acetate as the mobile phase of the silica gel column. Thus, the novel antibiotic having the above-mentioned properties can be obtained.

As a result of the examination of the antibacterial activity of the compound of Formula (I) or the compound having the above physico-chemical properties, especially excellent antibacterial activity was found with respect to a Geobacillus stearothermophilus ATCC 7953 strain, a Bacillus subtilis NBRC 3134^{T} strain, a Streptomyces griseus NBRC 15744^{T} strain, an Escherichia coli NBRC 102203^{T} strain, a Thermosporothrix hazakensis SK20-1^{T}/NBRC 105916^{T} strain, a Thermosporothrix narukonensis F4^{T} strain, a Streptomyces sp. AGRN-7 strain, a Streptomyces sp. AGRN-8 strain, and a Streptomyces sp. AGRN-9 strain.

The present invention also relates to an antibacterial agent containing a compound of Formula (I), or a compound having the above physico-chemical properties, or a salt or ester thereof as the active ingredient (hereinafter also referred to as an antibacterial agent of the present invention). The antibacterial agent of the present invention may be prepared for use in a form of composition by mixing a compound of Formula (I), or a compound having the above physico-chemical properties, or a salt or ester thereof as the active ingredient with a commonly used liquid or solid carrier, such as ethanol, water, and starch. There is no particular restriction on the effective concentration, insofar as an antibacterial property can be exserted, and it may be for example from 1 to 1000 μg/mL.

### EXAMPLES

The present invention will be described below more specifically with reference to Examples, provided that the present invention is not limited to these Examples.

### [Example 1] Purification of Antibacterial Active Substance “HK-1” Produced by Thermosporothrix hazakensis SK20-1^{T}

<Culture Strain Used>

Thermosporothrix hazakensis SK20-1^{T} NBRC 105916^{T}

<Culture Media Used>

TABLE 1
YS culture medium

| | | |
|---|---|---|
| Bacto ™ Yeast Extract | 2.0 g | Becton, Dickinson and Company (USA) |
| Soluble starch | 10.0 g | |
| Agar | 20.0 g | Shoei Kanten |

Total volume 1000 mL
Autoclave treatment at 121° C.
for 20 min

LB (+M) culture medium

| | |
|---|---|
| Bacto ™ Tryptone | 10.0 g |
| Bacto ™ Yeast Extract | 5.0 g |
| NaCl | 5.0 g |
| Maltose | 10.0 g |

Total volume 1000 mL
Autoclave treatment at 121° C.
for 20 min

R2A (+L) culture medium

| | | |
|---|---|---|
| Bacto ™ Peptone | 5.0 g | Becton, Dickinson and Company |
| Bacto ™ Yeast Extract | 5.0 g | |
| Bacto ™ Casamino | 5.0 g | Becton, Dickinson and Company |
| Acids, Technical | | |
| K₂HPO₄ | 3.0 g | |
| MgSO₄•7H₂O | 0.5 g | |
| Lactose | 10.0 g | |

pH set at 7.2
Total volume 1000 mL
Autoclave treatment at 121° C.
for 20 min

<Experimental Method>

1. Culture of T. hazakensis

1.1 Starter Culture

In a 300-mL Erlenmeyer flask 250 mL of a YS culture medium was prepared and subjected to an autoclave sterilization treatment (121° C., 20 min). After cooling down to 50° C. or lower, 25 mL each was aliquoted into a petri dish (10 plates were prepared). A piece of ice was taken out of a glycerol stock of T. hazakensis stored at −80° C. and inoculated on the YS culture medium in a dish in a radial pattern using a toothpick. This was cultured by static culture at 50° C. for 4 days.

1.2 Seed Culture

In a 500-mL Erlenmeyer flask with baffles 100 mL of LB (+M) culture medium was prepared and subjected to an autoclave sterilization treatment (121° C., 20 min). After cooling down to 50° C. or lower, 1 cm² of the inoculated in the “1.1 Starter culture” was cut out with a toothpick and T. hazakensis was inoculated thereon. This was cultured on a shaking incubator at 45° C. and 135 rpm for 24 hours.

1.3 Culture in Jar Fermenter

DIAION HP-20 (Nippon Rensui Co.) was added to two 4-L mini-jars (ABLE Corporation) and one 5-L mini-jar (Marubishi Bioengineering Co., Ltd.) in the respective amounts of 10 g and 15 g. In a 10-L plastic beaker, 7 L of R2A (+L) culture medium was prepared, and 2 L each was added to the 4-L mini-jars, and 3 L was added to the 5-L mini-jar, and they were subjected to an autoclave sterilization treatment (121° C., 15 min). After the sterilization treatment, they were left standing for 1 day to cool down the culture medium. The seed-cultured T. hazakensis was inoculated into them in an amount equivalent to 0.1% of that of the culture medium in each mini-jar. After connecting to the control device, air was introduced in an amount equivalent to ½ of that of each culture medium, and shaking culture was performed at 45° C. for 4 days at a rotation speed of 160 rpm for the 4-L mini jars, and 135 rpm for the 5-L mini-jar.

2. Extraction Experiment

2.1 Extraction

The total 28 L of the HP-20 supplemented medium in the respective mini-jars were combined and poured on a diameter 315/opening 200-μm sieve and a diameter 200/opening 300-μm sieve to recover the bacterial body and HP-20. The recovered material was aliquoted into four 500-mL centrifuge tubes, and the same amount of 100% acetone as the sample was added. After vigorous stirring, the mixture was shaken for 30 min in a reciprocating shaking incubator (135 rpm). The centrifuge tubes were centrifuged in a cooling centrifuge (4° C., 6,000 rpm, 10 min). The supernatant obtained by centrifugation was filtered with suction using a Kiriyama funnel, and the filtrate was transferred to a recovery flask (for evaporator) to distill off acetone in the sample with an evaporator. Total 2.8 L of the concentrate was stored in a room at 4° C.

2.2 Liquid-Liquid Partition

(1) A sample of an aqueous acetone solution extract was concentrated to 800 mL in an evaporator to completely remove acetone.

(2) The obtained sample was adjusted to pH 5.7 with 6 N hydrochloric acid.

(3) The whole amount was put into a 3,000-mL separating funnel, and 400 mL of ethyl acetate was added.

(4) The separating funnel was vigorously shaken for 15 to 30 sec, and the stopper was opened to vent the gas while keeping the opening upward.

(5) After repeating the operation of (4) above five to six times, the solution was allowed to stand until it separated.

(6) The lower layer and the upper layer were divided into 2,000-mL Erlenmeyer flasks, and the upper layer was used as an ethyl acetate extract.

(7) With respect to the lower layer, the same procedures as in (3) to (6) above were repeated twice.

(8) The total amount of the ethyl acetate extracts was combined and placed in a 3,000-mL separating funnel, and 500 mL of a 5% aqueous solution of sodium hydrogen carbonate was added thereto.

(9) After performing the operations of (4) to (5) above, the lower layer was used as an extract, and 500 mL thereof was taken.

(10) With respect to the upper layer, the same procedures as in (8) to (9) above were repeated.

(11) The pH of 1,000 mL of the obtained lower layer was adjusted to 5.7 with 6 N hydrochloric acid.

(12) The entire amount of the lower layer was placed in a 3,000-mL separating funnel, and the equal amount of ethyl acetate was added thereto.

(13) After performing the operations of (4) to (6) above, the upper layer was used as an extract and 500 mL thereof was taken.

(14) With respect to the lower layer, the same procedures as in (3) to (6) above were repeated twice.

(15) The total amount of the ethyl acetate extracts was combined, to which an appropriate amount of anhydrous sodium sulfate was added (to the extent that water disappeared and the extract became solid), and then left standing at room temperature for 15 min.

(16) The above was filtered with a filter paper, and exsiccated using an evaporator.

(17) The yield was measured using an electric balance.

3. Silica Gel Column Chromatography

Fifty (50) g of C-200 silica gel (Wakogel (R) C-200, FUJIFILM Wako Pure Chemical Corporation) was suspended in 100 mL of ethyl acetate, and poured into a column. The column was tapped from outside to drip the silica gel suspension. The exsiccated ethyl acetate extract was dissolved in 6 mL of ethyl acetate and placed into the column. Ethyl acetate was added to the top, and when the color of the target substance (yellow) reaches the bottom, it started receiving respectively 15-mL fractions of the eluate in test tubes. When the received fraction did not present the color any more, the solvent was changed to ethyl acetate/methanol=1/1 and the fractions of the eluate were received further until the eluate became colorless.

4. Thin Layer Chromatography

Every second fraction of the received ethyl acetate eluate was spotted on a TLC plate (Silica gel 60 F254, MERCK KGaA) using a capillary. After drying with a dryer, the plate was developed using a developing solution (ethyl acetate/acetic acid=4 mL/0.5 mL). After the developing solution reached about 1 cm from the top of the TLC plate, it was taken out from the developing layer and dried with a dryer. Spots were detected on the TLC plate under irradiation with UV 254 nm and UV 325 nm. A fraction with few contaminant spots was concentrated using an evaporator and then dried up in a desiccator. The yield was measured using an electric balance.

<Results>

When the 50% acetone extract of the HP-20 and the bacterial body added to the culture solution was concentrated and then extracted with ethyl acetate, 1.0 g of an extract was obtained. The obtained extract was subjected to silica gel column chromatography, and eluted with ethyl acetate. The eluate was fractionated, and each fraction was analyzed by TLC. As a result, a single spot (Rf=0.74, hereinafter referred to as HK-1) was confirmed in the 18th to 25th fractions. From the 18th to 25th fractions, 240 mg of HK-1 was obtained.

### [Example 2] Structural Analysis of HK-1

[1. Physical and Chemical Properties of HK-1]

<Experimental Method>

1. MS

A small amount of HK-1 was dissolved in methanol. It was subjected to an ESI(−)/TOF-MS spectral analysis. The analytical instruments are as follows.

Agilent 1110 series (pump)

The Accu TOF JMC-T100LC (MS)

2. HRMS

A request was made to the National Institute of Health Sciences for a measurement. The profile of (M-H)⁻ ion was analyzed with HRMS. The analytical instruments are as follows.

Agilent 1200 series (pump)

Agilent 6530 Q-TOF (MS)

3. UV

A small amount of HK-1 was dissolved in methanol and an absorption maximum was measured with a JASCO V-630 Spectrophotometer.

4. pH Stability

Into a 50-mL Falcon tube, 20 mg of HK-1 was weighed out using an electric balance, and therein 20 mL of methanol (1000 γ) was added followed by gentle stirring. Each 3.3 mL was aliquoted to totally two 15-mL Falcon tubes. To one of them 20 μL of 1 N sodium hydroxide was added followed by gentle stirring. The pH was tested with a pH test paper. Ten (10) μL of each sample was subjected to HPLC. The HPLC analysis conditions are as follows.

Column: CAPCELL PAK Cis 5 μm, 4.6 mm 1. D×150 mm

Mobile phase A: 0.1% aqueous solution of trifluoroacetic acid

Mobile phase B: acetonitrile

Temperature of thermostatic chamber for column: 40° C.

Flow velocity: 1 mL/min

UV: 254 nm

Elution method: concentration gradient
0 min to 18 min, B concentration 50% to 100%
18.1 min to 23 min, B concentration 100%
23.1 min to 25 min, B concentration 50%

<Results and Discussion>

HK-1 is a light yellow-colored powder, and its methanol solution also presents a similar color. In the UV spectrum of HK-1 measured with an ultraviolet-visible spectrophotometer, an absorption maximum was observed at 340 nm, and the molar absorption coefficient was 30630. In the ESI/TOF/MS spectrum measured in the negative mode, ions conceived as (M-H)⁻ ions were observed near m/z 1645, which signal pattern suggested that a halogen atom was involved. In addition, ions thought to be (M−2H)²⁻ ions were observed near m/z 822. Furthermore, as a result of profile analysis by HRMS of ions near m/z 1645, it was found that HK-1 is a compound of the molecular formula C₇₄H₅₈Cl₈O₂₆ having eight chlorine atoms. The solution of HK-1 prepared without adjusting the pH, and that prepared by adjusting the pH to 9 were found to be stable with no recognizable degradation peak in a HPLC analysis. The specific rotation [α]²⁴_{D} was −49 (c=1.0, DMSO).

[2. NMR Spectrum of HK-1]

<Experimental Method>

Eighty (80) mg of HK-1 was dissolved in 0.75 mL of dimethyl sulfoxide-d₆, 99.9% D (for NMR, Kanto Chemical Co., Ltd.). A solution with dissolved HK-1 was sent through a Pasteur pipette filled with silica wool and placed into an NMR tube. The tube was closed with a cap, sealed hermetically with a parafilm, and analyzed in a shaded condition (NMR instrument, Varian Inova 500).

<Results and Discussion>

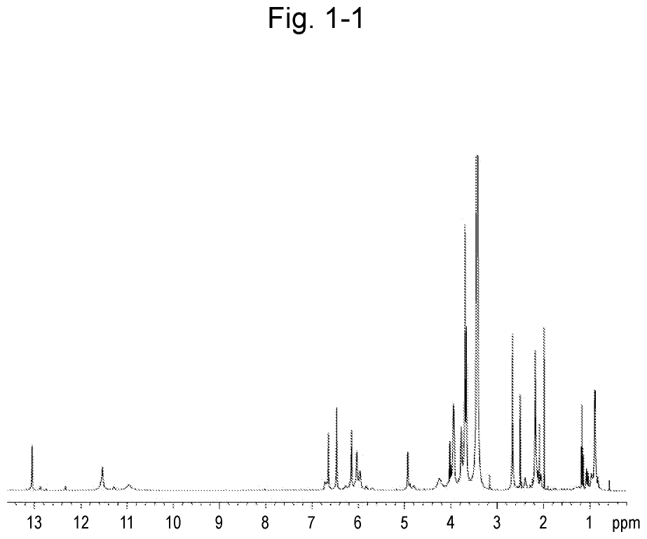

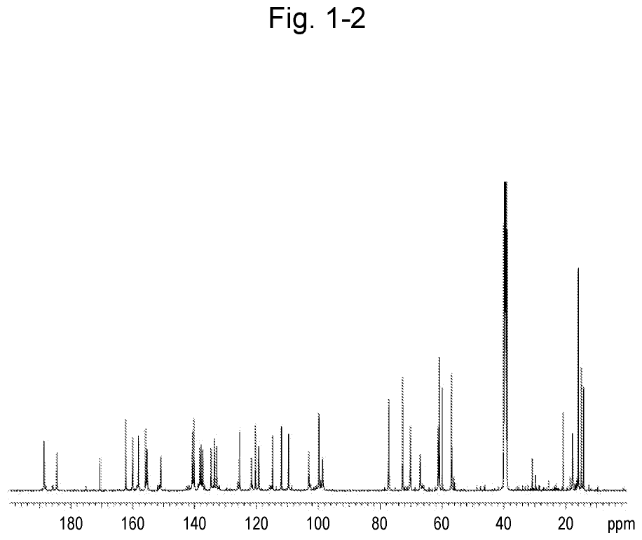

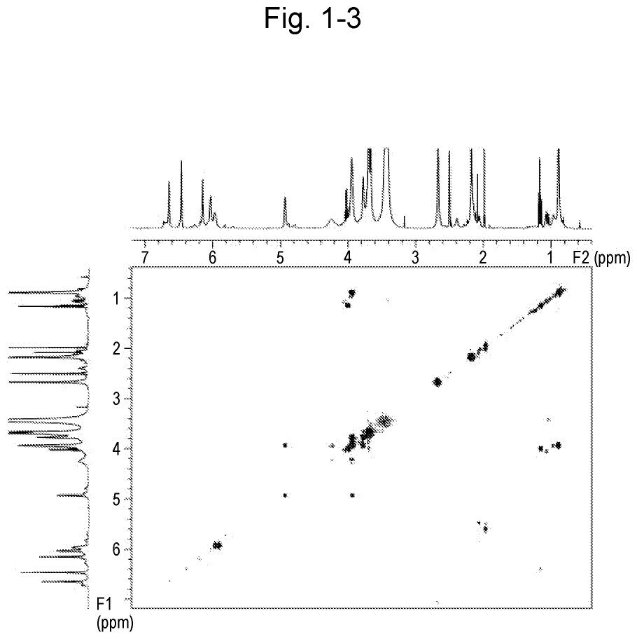

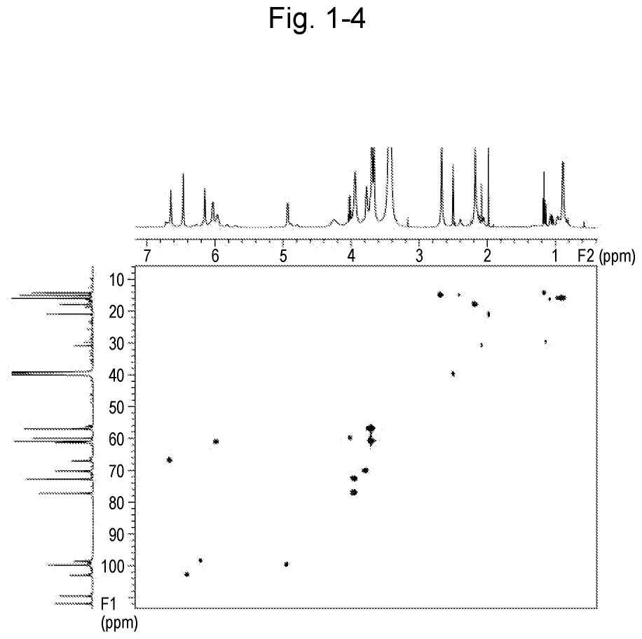

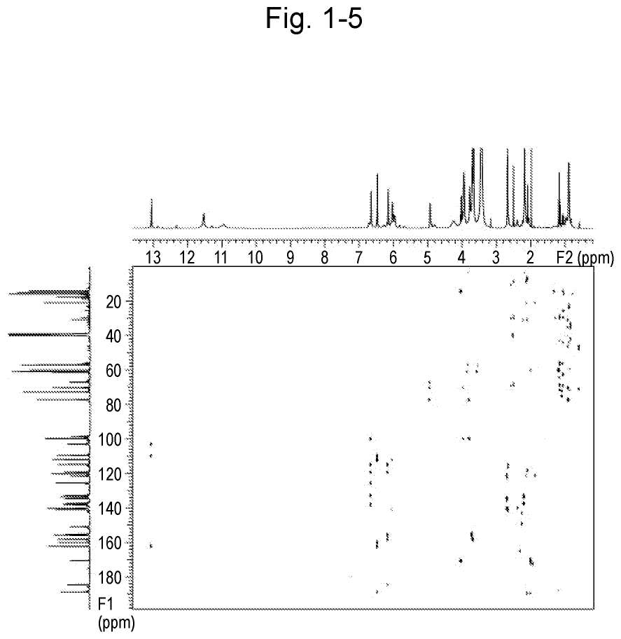

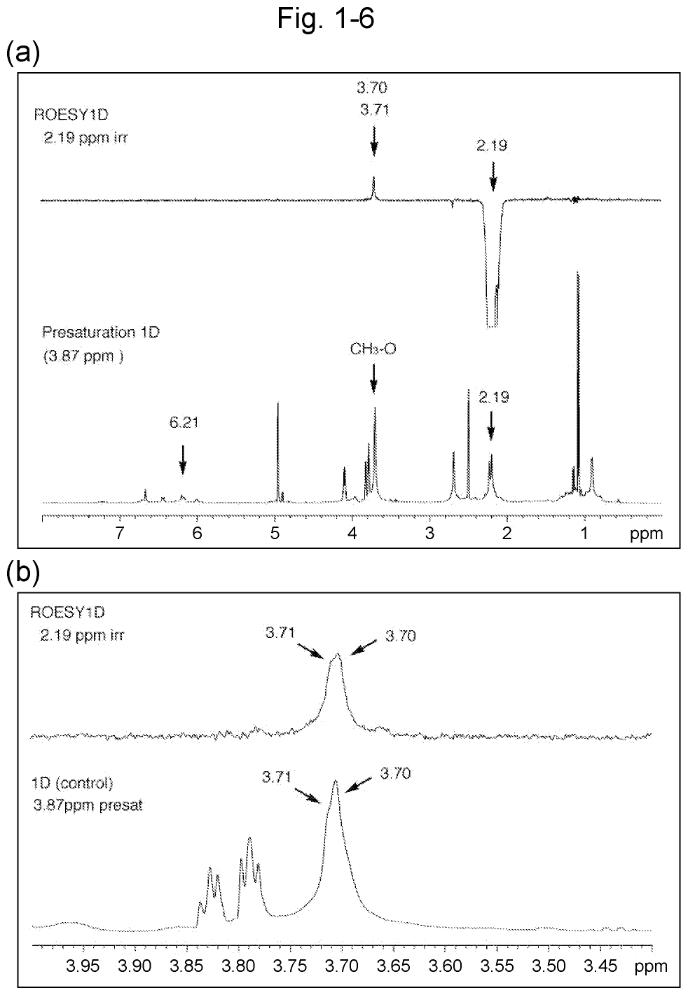

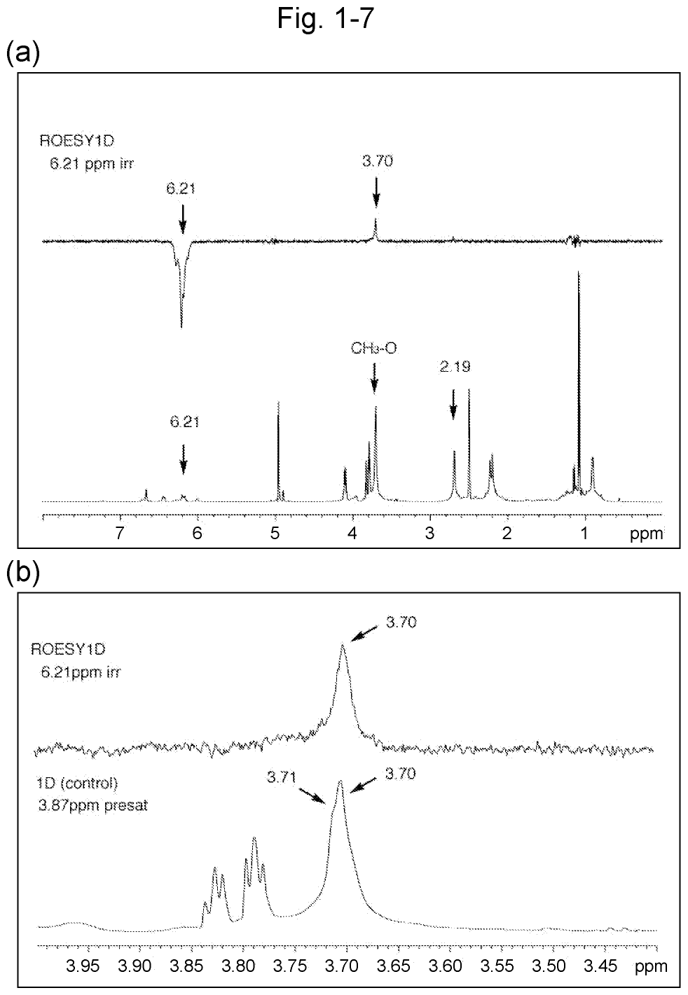

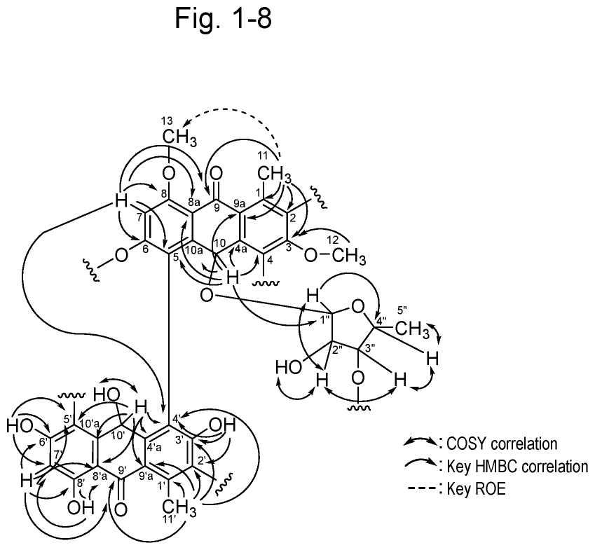

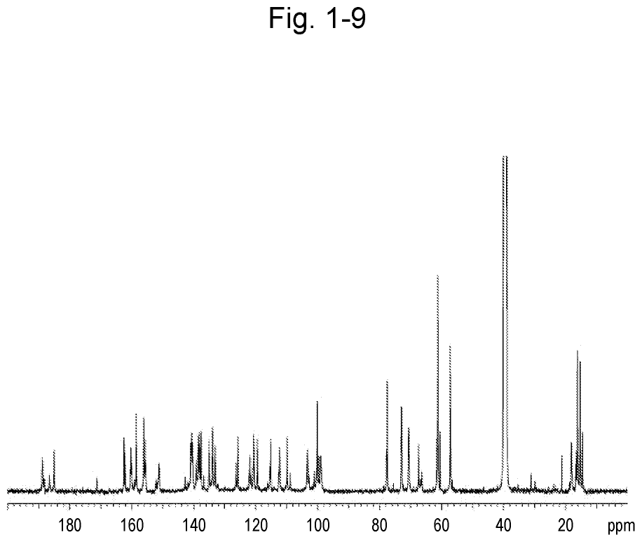

The spectra of ¹H-NMR, ¹³C-NMR, COSY, HSQC, and HMBC with respect to HK-1 are shown in FIG. 1-1 to FIG. 1-5. Furthermore, the 1D-ROESY spectra of HK-1 irradiated at 2.19 ppm or 6.21 ppm are shown in FIG. 1-6 and FIG. 1-7, respectively. In the 1D-ROESY spectrum (FIG. 1-6), ROE was observed at the methoxy proton (H-13 position) when irradiated at the H-11 position (2.19 ppm), so the position of the methoxy group (C-13 position) was determined as C-8 position. From these NMR spectral analyses, it was made clear that HK-1 had the partial structure shown in FIG. 1-8. Since this partial structure includes all of 37 carbon signals observed in the ¹³C-NMR spectrum, it was suggested that HK-1 has a structure in which this partial structure forms a dimer.

[3. Hydrolysis Reaction with Hydrochloric Acid]

<Experimental Method>

1. Hydrolysis

One (1) mg of HK-1 was placed in a 20-mL recovery flask with a stirrer bar. After the addition of 1 mL of acetonitrile, 1 mL of 1 M hydrochloric acid was slowly added in portions of a few drops with stirring. The mixed liquid was heated at reflux for 30 min on an oil bath, returned to room temperature, and then freeze-dried

2. HRMS

A request was made to the National Institute of Health Sciences for a measurement with respect to the hydrolysate with hydrochloric acid after freeze-drying. The profile near the (M-H)⁻ ion was analyzed in HRMS. The analytical instruments are as follows.

Agilent 1200 series (pump)

Agilent 6530 Q-TOF (MS)

<Results and Discussion>

HK-1 was hydrolyzed in 50% acetonitrile containing hydrochloric acid at a concentration of 0.5 M by heating at reflux, and then the hydrolysate was analyzed as it was by Q-TOF/MS in the negative mode. In the obtained mass spectrum, ions with the maximum intensity at m/z 1413 were observed, and HRMS revealed that the molecular formula of the hydrolysate was C₆₄H₄₂Cl₈O₂₀. This molecular formula of the hydrolysate was identical with that of aglycone formed when the two 5-deoxyfuranosides of HK-1 were removed by hydrolysis. Therefore, it was shown that a hydroxy group is bonded to the carbon at position 3 of 5-deoxyfuranoside of HK-1.

[4. ¹³C-NMR Spectral Analysis]

From the analyses above, it was considered that HK-1 has a structure in which the partial structure in FIG. 1-8 having a hydroxy group at C-3″ is dimerized. When four chlorine atoms are added to the monomer part, the molecular formula of the monomer structure part of HK-1 is satisfied. In FIG. 1-8, the carbons to which a chlorine atom can bind are C-2, C-4, C-6, C-2′ and C-5′, however since oxygen binds to C-6, it has become clear that chlorine atoms bind to C-2, C-4, C-2′ and C-5′.

[5. Confirmation of Binding Position of Hydroxy Group]

The binding positions of six hydroxy groups present in HK-1 were confirmed by using the method in which the carbon having a hydroxy group is determined based on the fact the chemical shift in the ¹³C-NMR spectrum of the carbon having a hydroxy group is different in D₂O and H₂O due to isomer shift. When measured using a duplex NMR tube containing D₂O and H₂O solutions respectively, the ¹³C-NMR spectrum shown in FIG. 1-9 was obtained, and two signals were observed at C-3′, C-6′, C-8′, C-10′, C-2″ and C-3″ each, to which respective hydroxy groups bind, to confirm the binding positions of six hydroxy groups.

[6. Structure of HK-1]

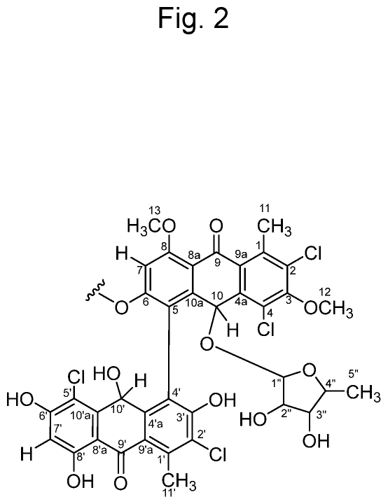

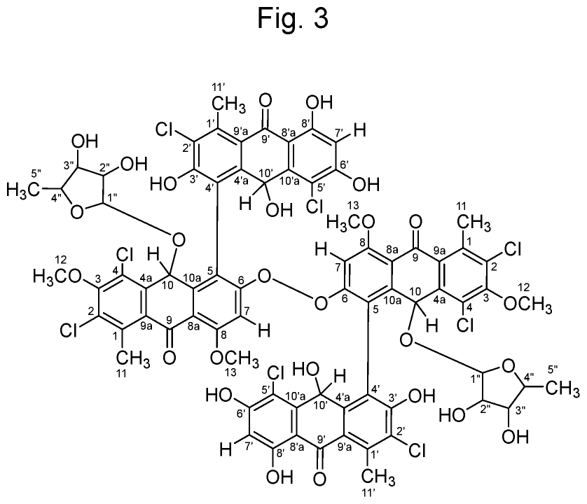

From the above, it was known that HK-1 has a dimeric structure shown in FIG. 3, in which a peroxide is formed between the oxygens each of which is at the C-6 position of the monomer structure shown in FIG. 2. Table 2 shows the assignment of each signal in the ¹H-NMR and ¹³C-NMR spectra. There are few natural products having a diphenyl peroxide structure and it is only known in bungein A isolated as a plant component (Hui Yang, Ai-Jun Hou, Shuang-Xi Mei, Han-Dong Sun, and Chun-Tao Che, 2002, Constituents of Clerodendrum Bungei, Journal of Asian Natural Products Research, 4, 165-169). It was reported based on the chemical shifts of 5 carbons of 5-deoxyfuranoside (99.78 (C-1″), 72.75 (C-2″), 70.21 (C-3″), 77.15 (C-4″), and 15.90 (C-5″)), that 5-deoxyl-β-lyxofuranose form a glycosidic linkage at the C-10 position (Joseph R, Snyder, and Anthony S. Serianni, 1987, Synthesis and N.M.R.-spectral analysis of unenriched and [1-¹³C]-enriched 5-deoxypentoses and 5-O-methylpentoses, Carbohydrate Research, 163 (1987) 169-188). There has been no report on 5-deoxylyxose as a natural product, and HK-1 is the first case.

TABLE 2
Assignment of signals in ¹H-NMR and ¹³C-NMR spectra of HK-1^{a}

| C-No | δ_{C} | δ_{H} | HMBC (H→C) |
|---|---|---|---|
| 1 | 137.26^{b} | | |
| 2 | 133.53^{b} | | |
| 3 | 155.25 | | |
| 4 | 125.42^{c} | | |
| 4a | 137.79^{c} | | |
| 5 | 114.71^{d} | | |
| 6 | 155.76 | | |
| 7 | 98.55 | 6.21 | 5, 6, 8, 8a, 4′ |
| 8 | 158.11 | | |
| 8a | 119.19^{d} | | |
| 9 | 184.49 | | |
| 9a | 132.77 | | |
| 10 | 67.00 | 6.67 | 4, 4a, 5, 8a, 9a, 10a, 1″ |
| 10a | 138.20^{c} | | |
| 11 | 17.83 | 2.19 | 1, 2, 3, 4 or 4a, 9, 9a |
| 12 | 60.68 | 3.71 | 3 |
| 13 | 56.94 | 3.70 | 8 |
| 1′ | 134.60 | | |
| 2′ | 140.25 | | |
| 3′ | 150.91 | | |
| 3′-OH | | 10.92 | 2′, 3′, 4′ |
| 4′ | 120.24 | | |
| 4′a | 140.25^{e} | | |
| 5′ | 111.89^{f} | | |
| 6′ | 159.96^{f} | | |
| 6′-OH | | 11.49 | 5′, 6′, 7′ |
| 7′ | 102.99 | 6.41 | 5′, 6′, 8′, 8′a, 9′ |
| 8′ | 162.20^{f} | | |
| 8′a | 109.54^{f} | | |
| 9′ | 188.61 | | |
| 9′a | 121.51 | | |
| 10′ | 61.20 | 6.00 | 4′, 4′a, 5′, 8′a, 9′a, 10′a |
| 10′a | 140.73^{e} | | |
| 10′-OH | | 5.91 | |
| 11′ | 14.96 | 2.69 | 1′, 2′, 3′, 4′, 9′, 9′a |
| 1″ | 99.78^{g} | 4.95^{h} | 3″, 4″, 10 |
| 2′ | 72.75 | 3.95 | |
| 2″-OH | | 4.26 | |
| 3″ | 70.21 | 3.79 | 4″ |
| 4″ | 77.15 | 3.96 | |
| 5″ | 15.90 | 0.90ⁱ | 3″, 4″ |

^{a}The spectrum was measured in DMSO-d₆.
^{b, c, d, e}Interchangeable
^{f}The combination of 111.89 and 159.96 is exchangeable with the combination of 109.54 and 162.20.
^{g}J_{C-1″}, _{H-1″} = 172.5 Hz
^{h}J_{H-1″}, _{H-2″} = 4.7 Hz
ⁱJ_{H-5″}, _{H-4″} = 5.7 Hz

### [Example 3] Test for Measuring HK-1 Activity

[1. Antibacterial Spectrum Test]

The antibacterial activity of HK-1 was assayed using a paper disk against the following bacterial bodies.

<Strains Used>

Geobacillus stearothermophilus ATCC 7953

Bacillus subtilis NBRC 3134^{T}

Streptomyces griseus NBRC 15744^{T}

Escherichia coli NBRC 102203^{T}

Thermosporothrix hazakensis SK20-1^{T} NBRC 105916^{T}

Thermosporothrix narukonensis F4^{T}

Streptomyces sp. AGRN-7

Streptomyces sp. AGRN-8

Streptomyces sp. AGRN-9

<Culture Media Used>

TABLE 3
YMPD culture medium

| | |
|---|---|
| Fish extract for bacteria | 2.0 g |
| Bacto ™ Yeast Extract | 2.0 g |
| Bacto ™ Peptone | 4.0 g |
| MgSO₄•7H₂O | 2.0 g |
| NaCl | 5.0 g |
| D(+)-glucose | 10.0 g |
| Agar | 15.0 g |

pH set at 7.2
Total volume 1000 mL
Autoclave treatment at 121° C.
for 20 min

ISP2 culture medium

| | | |
|---|---|---|
| Bacto ™ Yeast Extract | 4.0 g | |
| Bacto ™ Malt Extract | 10.0 g | Becton, Dickinson and Company |
| D(+)-glucose | 4.0 g | |
| Agar | 20.0 g | |

pH set at 7.3
Total volume 1000 mL
Autoclave treatment at 121° C.
for 20 min

BM culture medium

| | | |
|---|---|---|
| Fish extract for bacteria | 1.0 g | Kyokuto Pharmaceutical Industrial |
| | | Co., Ltd. |
| Bacto ™ Yeast Extract | 1.0 g | |
| NZ Amine | 2.0 g | Wako Pure Chemical Industries, Ltd. |
| Maltose | 10.0 g | |
| Agar | 15.0 g | |

Total volume 1000 mL
Autoclave treatment at 121° C.
for 20 min

<Experimental Method>

1. Antibacterial Assay with G. stearothermophilus

A piece of ice was taken out of a glycerol stock of G. stearothermophilus stored at −80° C. and planarly inoculated on the LB agar culture medium (see Example 1) with a toothpick. This was incubated at 60° C. for 18 hours. After the incubation, ½ platinum loop of the culture was harvested and suspended in 500 μL of sterile water. The bacterial suspension was diluted 10-fold with sterile water. Onto a LB agar culture medium, 100 μL of the diluted suspension was dropped and spread until it was dried up. A piece of paper disk was placed on a filter paper, onto which 50 μL of a HK-1 solution adjusted to 1000 γ with methanol was dropped and dried. Then the disk was placed on the agar culture medium coated with the bacterial body. After incubation at 60° C. for 18 hours, the diameter of the inhibition ring was measured.

2. Antibacterial Assay with B. subtilis, or E. coli

Assays were performed in the same manner as in 1 above, except that the incubation temperature was changed to 37° C.

3. Antibacterial Assay with S. griseus

A piece of ice out of a glycerol stock of S. griseus stored at −80° C. was planarly inoculated on the YMPD agar culture medium with a toothpick. This was incubated at 28° C. for 5 days. After the incubation, a small amount of spore was scraped off with a swab, and planarly inoculated on the YMPD culture medium. A piece of paper disk was placed on a filter paper, onto which 50 μL of a HK-1 solution adjusted to 1000 γ with methanol was dropped and dried. Then the disk was placed on the agar culture medium coated with the bacterial body. After incubation at 28° C. for 4 days, the diameter of the inhibition ring was measured.

4. Antibacterial Assay with T. hazakensis, or T. narukonensis

Assays were performed in the same manner as in 3 above, except that the incubation conditions were changed to YS culture medium (see Example 1), 50° C., and 2-day period.

5. Antibacterial Assay with Streptomyces sp. AGRN-7

An assay was performed in the same manner as in 3 above, except that the incubation conditions were changed to ISP2 culture medium, 45° C., and 2-day period.

6. Antibacterial Assay with Streptomyces sp. AGRN-8, or 9

Assays were performed in the same manner as in 3 above, except that the incubation conditions were changed to BM culture medium, 45° C., and 2-day period.

<Results and Discussion>

The respective inhibition ring diameters for the Gram-positive bacteria were 32.0 mm for B. subtilis, 34.0 mm for G. stearothermophilus, 38.0 mm for S. griseus, 10.0 mm for the HK-1 producing bacterium of T. hazakensis, and also 10.0 mm for its relative strain of T. narukonensis. In addition, the inhibition rings were formed with a diameter of 22.0 mm for Streptomyces sp. AGRN-7 (its 16S rRNA gene sequence has similarity of 99% to Streptomyces thermodiastaticus), which is a thermophilic actinomycete isolated from the compost of AGORA Landscape Architecture Corporation, 16.0 mm for Streptomyces sp. AGRN-8 (its 16S rRNA gene sequence has similarity of 99% to Streptomyces mexicanus), and 19.0 mm for Streptomyces sp. AGRN-9 (its 16S rRNA gene sequence has similarity of 99% to Streptomyces leeuwenhoeckii). For E. coli, which is a gram-negative bacterium, an inhibition ring with a diameter of 14.0 mm was formed

From the size of the inhibition ring, it was shown that HK-1 has a high antibacterial activity against Gram-positive bacteria. It was also found that it exhibits antibacterial activity against various thermophilic actinomycetes. It is known that actinomycetes that produce antibiotics have high resistance to a drug, but from the above results it was shown that HK-1 is also an effective antibiotic substance against such actinomycetes. Since HK-1 at a high concentration exhibits an antibacterial action against the T. hazakensis itself, which is a HK-1-producing bacterium, it was conceived that the HK-1-producing bacterium possibly controls the production amount of HK-1 below the lethal concentration.

[2. Measurement of Minimum Inhibitory Concentration]

The minimum inhibitory concentration (MIC) is one of the antibiotic susceptibility tests, and means the minimum concentration of an antimicrobial substance at which the visible growth of a microorganism can be inhibited. In the research field, the MIC measurement is used, for example, to compare the effects of a new antibiotic substance and a conventional substance, or to examine actions on several strains, and is regarded as an important criterion for evaluating antibiotic substances. The MIC of “HK-1” which was obtained hereunder as the antibacterial active substance was measured using the dilution method with a liquid culture medium.

<Strains Used>

Geobacillus stearothermophilus 111499 MERCK (ATCC 7953)

Bacillus subtilis NBRC 3134^{T}

<Culture Medium Used>

TABLE 4
LB culture medium

| | |
|---|---|
| Bacto ™ Tryptone | 10.0 g |
| Bacto ™ Yeast Extract | 5.0 g |
| NaCl | 5.0 g |
| Agar | 20.0 g |

Total volume 1000 mL
Autoclave treatment at 121° C.
for 20 min

<Experimental Method>

1. Preparation of G. stearothermophilus Bacterial Suspension

A piece of ice out of a glycerol stock of G. stearothermophilus stored at −80° C. was planarly inoculated on the LB agar culture medium with a toothpick. This was incubated at 60° C. for 18 hours. One (1) cm² of the culture medium was excavated with a toothpick, placed into a long test tube containing 10 mL of a liquid LB culture medium, and subjected to a shaking culture at 60° C., and 300 rpm, for 18 hours. The culture solution was diluted with sterile water to about McF standard No. 1.

2. Preparation of B. subtilis Bacterial Suspension

The preparation was performed in the same manner as in 1 above, only except that the temperature was changed to 37° C.

3. Preparation of HK-1-Supplemented Medium

Two (2) mg of HK-1 was weighed out into a 2 mL Eppendorf tube using an electric balance. To this Eppendorf tube, 2 mL of methanol was added to dissolve the HK-1 to form a 10 kγ solution. The solution was filtrated through a sterilized 0.22-nm filter. Using 100 μL of the HK-1 solution, a 50 μL 2-fold dilution series (from 10 kγ to 19 γ) was prepared using methanol. One (1) % each was added to a test tube containing 5 mL of a LB liquid culture medium. One (1) % of each of the culture solutions prepared in 1 and 2 above was added to the test tube. Incubations were performed at the respective temperatures (G. stearothermophilus: 60° C., B. subtilis: 37° C.) and 135 rpm for 18 hours. After the incubations, visual observation and measurement of OD₆₀₀ were performed using a test tube in which 1% methanol and sterile water were added to 5 mL of the LB liquid culture medium as a control.

<Results and Discussion>

HK-1 inhibits growth of both strains down to a concentration of 0.78 μg/mL. From the OD₆₀₀ measurement results, there was no significant difference in the test tubes of 0.78 μg/mL or less in which turbidity was observed. A culture medium to which HK-1 was added at a high concentration gave a slightly yellowish tint compared to an ordinary LB culture medium. The color did not change after incubation.

From the above results, it was shown that HK-1 has a relatively strong antibacterial effect on Bacillus subtilis, and G. stearothermophilus, which is a food spoilage causing bacterium.

[3. Antibacterial Activity Test in Changing pH]

The stability of a compound is regarded as one of the important information for evaluating a substance. This is because, even if the external environment changes, insofar as the compound itself is stable, it can be applied in various situations, and the information leads to acquisition of biological knowledges. Therefore, the stability of the activity was examined by measuring antibacterial activities with respect to G. stearothermophilus, and B. subtilis using the solution prepared in the above “1. Antibacterial spectrum test”.

<Strains Used>

Geobacillus stearothermophilus ATCC 7953

Bacillus subtilis NBRC 3134^{T}

<Culture Media Used>

TABLE 5
AM2 culture medium

| | | |
|---|---|---|
| Beef Extract | 1.5 g | |
| Bacto ™ Yeast Extract | 3.0 g | |
| Bacto ™ Peptone | 6.0 g | |
| Agar | 15.0 g | Ina Food Industry Co., Ltd. |

Total volume 1000 mL
Autoclave treatment at 121° C.
for 20 min

NB culture medium

| | | |
|---|---|---|
| Difco ™ Nutrient Broth | 8.0 g | Becton, Dickinson and Company |
| Agar | 5.0 g | |

Total volume 1000 mL
Autoclave treatment at 121° C.
for 20 min

<Experimental Method>

1. Preparation of Culture Medium for Assaying Antibacterial Activity, and Assay

1.1 Culture Medium for Assaying Antibacterial Activity Against Bacillus subtilis (for 10 Dishes)

<Preparation of Suspension of Bacillus subtilis Spore>

(1) In a 300-mL Erlenmeyer flask, 250 mL of a LB culture medium was prepared and sterilized in an autoclave (121° C., 20 min).

(2) After cooling down to 50° C. or lower, 25 mL each was dispensed into a petri dish (10 dishes were prepared).

(3) Each 30 μL of a B. subtilis spore suspension was planarly inoculated with a swab over 10 sheets of the LB agar culture medium (see Example 1).

(4) These were subjected to static culture at 37° C. for 1 week.

(5) Three (3) mL of sterile water was added to a piece of sterile absorbent cotton, and the spore in a dish was scraped off therewith.

(6) About 3 mL of the bacterial suspension was sucked up from the absorbent cotton with a 1-mL syringe, and transferred to a sterilized 50-mL Falcon tube.

(7) The above (5) and (6) were performed totally 10 times.

(8) The 50-mL Falcon tube containing about 30 mL of the bacterial suspension was heated in a bath at 60° C. for 30 min.

(9) Centrifugation was performed with a cooled centrifuge (6,000 rpm, 10 min, 4° C.), and then the supernatant was discarded.

(10) The precipitate and 30 mL of sterile water added were mixed with stirring.

(11) The above (9) and (10) were performed twice totally.

(12) Centrifugation was performed with a cooled centrifuge (6,000 rpm, 10 min, 4° C.), the supernatant was discarded, and then a small amount of sterile water was added only enough to dissolve the precipitate.

(13) The mixture was heated in a bath at 60° C. for 30 min.

(14) This was stored at 4° C., and regarded as a B. subtilis spore suspension.

<Preparation of Culture Medium for Assaying Antibacterial Activity Against Bacillus subtilis>

(1) In a 200-mL Erlenmeyer flask, 100 mL of a NB culture medium was prepared and sterilized in an autoclave (121° C., 20 min).

(2) After cooling to 50° C. or lower, 100 μL of the B. subtilis spore suspension was added.

(3) After gentle stirring, 10 mL each was dispensed into a petri dish.

(4) After solidification, this was stored at 4° C.

During the assay, incubation was performed at 37° C. for 18 hours.

1.2 Culture Medium for Assaying Antibacterial Activity Against Geobacillus stearothermophilus (for 10 Dishes)

(1) Each 10 mL of the (autoclave treated) LB culture medium (see Example 1) was dispensed into two long test tubes.

(2) Into each long test tube, 50 μL of a commercially available G. stearothermophilus spore suspension was seeded.

(3) Each long test tube was subjected to shaking culture at 60° C., and 300 rpm for 24 hours.

(4) In a 200-mL Erlenmeyer flask, 100 mL of an AM2 culture medium was prepared and sterilized in an autoclave (121° C., 20 min).

(5) After cooling down the above culture medium to 50° C. or lower, 20 mL of the G. stearothermophilus culture solution was added.

(6) After gentle stirring, 10 mL each thereof was dispensed into a petri dish.

(7) After solidification, it was stored at 4° C.

During the assay, incubation was performed at 50° C. for 18 hours.

1.3 Method of Measuring Activity of Each Culture Medium for Assaying Antibacterial Activity: Paper Disk Assay

A paper disk soaked with 50 μL of a sample was placed on an overlay medium with each bacterial body (B. subtilis, and G. stearothermophilus). The culture medium was incubated. The culture medium was taken out from the incubator, and the diameter of the inhibition ring was measured with calipers (Kanon hardened stainless 150 mm).

<Results and Discussion>

With respect to B. subtilis, the inhibition ring diameter of the solution for which the pH was not adjusted was 18.5 mm, while that of the solution for which the pH was adjusted to 9 was 20.2 mm. With respect to G. stearothermophilus, the inhibition ring diameter of the solution for which the pH was not adjusted was 21.9 mm, while that of the solution for which the pH was adjusted to 9 was 22.6 mm. The inhibition ring was slightly larger in a solution for which the pH was adjusted to be basic compared to the control.

[4. Stability Test During Temperature Fluctuation]

Examples of a typical test for measuring the stability of a compound include pH stability test, as well as heat resistance test, and storage test. This information is also regarded as important in evaluating the compound as described above. Using the sample of HK-1 prepared in the above “3. Antibacterial activity test in changing pH”, experiments were carried out to examine whether the compound is stable or not using G. stearothermophilus and B. subtilis guided by the antibacterial activity as the index.

<Strains Used>

Geobacillus stearothermophilus ATCC 7953

Bacillus subtilis NBRC 3134^{T}

<Experimental Method>

1. Heat Resistance Test A

Three hundred (300) μL of the 1000 γ HK-1 solution (pH 9, and pH not adjusted) prepared in the above “3. Antibacterial activity test in changing pH” was dispensed into a 1 mL microtube. The opening of the tube was closed hermetically with parafilm and heated in the bath at 98° C. for 5 min. An assay was performed using the culture medium for assaying antibacterial activity prepared in “3. Antibacterial activity test in changing pH” above.

2. Heat Resistance Test B

One (1) mL each of two samples prepared in the above “3. Antibacterial activity test in changing pH” was dispensed into two 1-mL micro tubes. The openings of total four tubes were closed hermetically with parafilm, and each one is shielded with an aluminum foil, and stored in a freezer set at −28° C., or an incubator set at 28° C. and 50° C. Each sample was assayed 1, 3, 6, 12, and 24 days after the initiation of the storage using the culture medium for assaying antibacterial activity prepared in “3. Antibacterial activity test in changing pH” above.

<Results and Discussion>

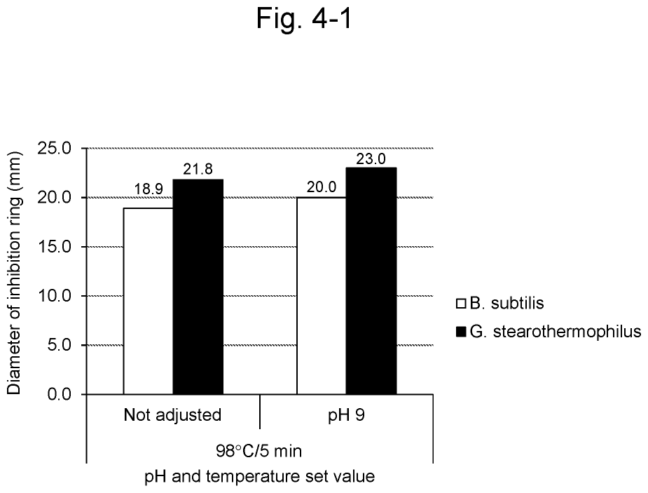

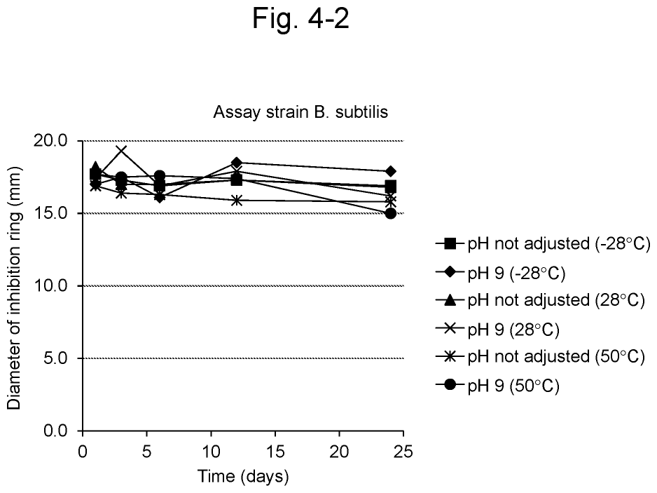

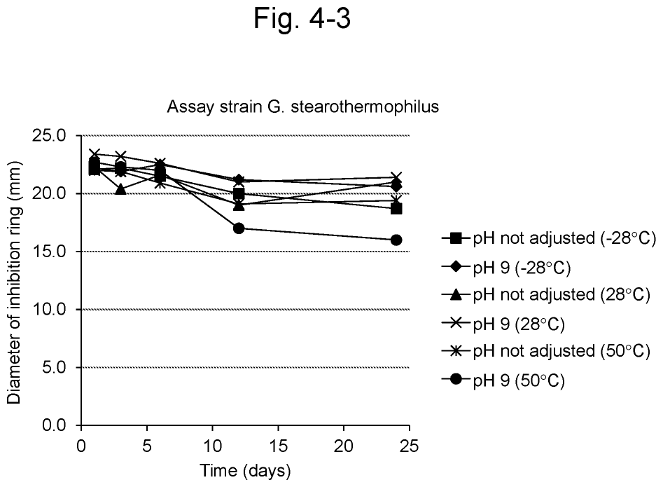

Regarding the heat resistance test, A, when pH was adjusted to 9 and HK-1 was heated at a high temperature, although there was some difference in the numerical values between the two samples, the antibacterial effect was not lost (FIG. 4-1).

Regarding the heat resistance test B, the antibacterial activity of two samples was not lost even after 24 days, indicating that the compound is resistant to high temperatures. Even with respect to HK-1, which pH was not adjusted, no significant difference was observed due to the storage temperature (FIG. 4-2 and FIG. 4-3).

### INDUSTRIAL APPLICABILITY

The compound of the present invention is useful as an antibacterial agent.

All publications, patent and patent applications cited herein are incorporated herein by citation in their entirety.

1. A compound represented by Formula (I): 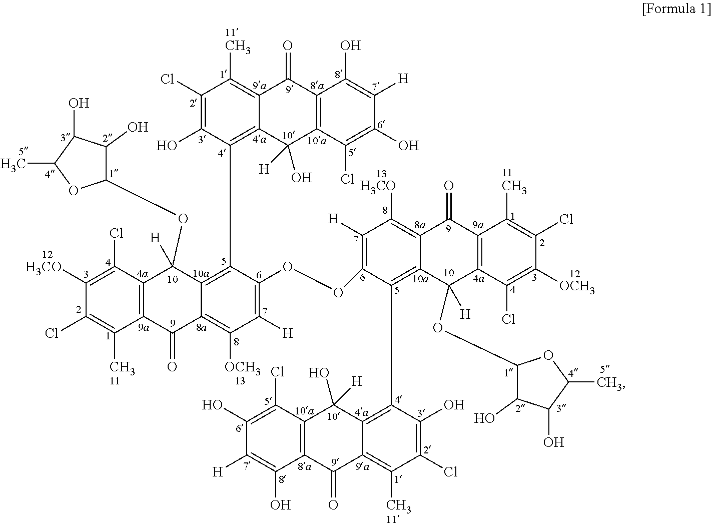 [SMILES: COc1cc(OOc2cc(OC)c3c(c2-c2c(O)c(Cl)c(C)c4c2C(O)c2c(Cl)c(O)cc(O)c2C4=O)C(OC2OC(C)C(O)C2O)c2c(Cl)c(OC)c(Cl)c(C)c2C3=O)c(-c2c(O)c(Cl)c(C)c3c2C(O)c2c(Cl)c(O)cc(O)c2C3=O)c2c1C(=O)c1c(C)c(Cl)c(OC)c(Cl)c1C2OC1OC(C)C(O)C1O]
or a salt or ester thereof.

2. A compound having an absorption maximum at 340 nm in its UV spectrum, and a molar absorption coefficient of 30630;
having a molecular formula of C₇₄H₅₈Cl₈O₂₆ as determined by analyses of an ESI/TOF/MS spectrum and a high-resolution mass spectrum;
showing signals at δ11.49, 10.92, 6.67, 6.41, 6.21, 6.00, 5.91, 4.95, 4.26, 3.96, 3.95, 3.79, 3.71, 3.70, 2.69, 2.19, and 0.90 in a ¹H NMR spectrum (DMSO-d₆); and
showing signals at δ188.61, 184.49, 162.20, 159.96, 158.11, 155.76, 155.25, 150.91, 140.73, 140.25, 140.25, 138.20, 137.79, 137.26, 134.60, 133.53, 132.77, 125.42, 121.51, 120.24, 119.19, 114.71, 111.89, 109.54, 102.99, 99.78, 98.55, 77.15, 72.75, 70.21, 67.00, 61.20, 60.68, 56.94, 17.83, 15.90, and 14.96 in a ¹³C NMR spectrum (DMSO-d₆);
or a salt or ester thereof.

3. The compound, or a salt or ester thereof according to claim 2, obtained by culturing Thermosporothrix hazakensis in a culture medium, and recovering the compound, or a salt or ester thereof from the culture product.

4. The compound, or a salt or ester thereof according to claim 3, wherein the compound, or a salt or ester thereof is recovered by treating an ethyl acetate extract with a silica gel column, the ethyl acetate extract being obtained by partitioning an aqueous acetone solution extract of the culture product between ethyl acetate and water.

5. The compound, or a salt or ester thereof according to claim 3, wherein Thermosporothrix hazakensis is a Thermosporothrix hazakensis SK20-1^{T} strain.

6. A method of producing the compound, or a salt or ester thereof according to claim 1, comprising a step of culturing Thermosporothrix hazakensis in a culture medium, and recovering the compound, or a salt or ester thereof from the culture product.

7. The method according to claim 6, wherein the compound, or a salt or ester thereof is recovered by treating an ethyl acetate extract with a silica gel column, the ethyl acetate extract being obtained by partitioning an aqueous acetone solution extract of the culture product between ethyl acetate and water.

8. The method according to claim 6, wherein Thermosporothrix hazakensis is a Thermosporothrix hazakensis SK20-1^{T} strain.

9. An antibacterial agent comprising the compound, or a salt or ester thereof according to claim 1, as an active ingredient.

10. An antibacterial agent comprising the compound, or a salt or ester thereof according to claim 2, as an active ingredient.

11. A method of inhibiting bacterial growth, comprising contacting a bacteria with an effective amount of the compound, or a salt or ester thereof of claim 1.

12. A method of inhibiting bacterial growth, comprising contacting a bacteria with an effective amount of the compound, or a salt or ester thereof of claim 2.

13. The method of claim 11, wherein the bacteria is selected from the group consisting of: Geobacillus stearothermophilus, Bacillus subtilis, Streptomyces griseus, Escherichia coli, Thermosporothrix hazakensis, Thermosporothrix narukonensis, and Streptomyces sp.

14. The method of claim 12, wherein the bacteria is selected from the group consisting of: Geobacillus stearothermophilus, Bacillus subtilis, Streptomyces griseus, Escherichia coli, Thermosporothrix hazakensis, Thermosporothrix narukonensis, and Streptomyces sp.
